(19) United States
(12) Patent Application Publication
(10) Pub. No.: US 2005/0107655 A1
(43) Pub. Date: May 19, 2005

(54) Method and apparatus for the prevention of epileptic seizures

(76) Inventor: Oliver Holzner, Bruck (DE)

Correspondence Address: HOUSTON ELISEEVA, 4 MILITIA DRIVE, SUITE 4, LEXINGTON, MA, 02421, US

(21) Appl. No.: 10/958,842

(22) Filed: Oct. 5, 2004

Related U.S. Application Data
(63) Continuation of application No. PCT/EP03/03543, filed on Apr. 4, 2003.

(30) Foreign Application Priority Data
Apr. 5, 2002 (DE) DE 102 15 115.6

(51) Int. Cl. A61N 2/00

(52) U.S. Cl. 600/9

(57) ABSTRACT

A method and an apparatus for automatic non-invasive controlled or regulated, respectively, electromagnetic prevention of epileptic seizures in vivo, based on seizure models is disclosed. Firstly the method comprises (in addition to the ongoing measurement of electromagnetic fields, in particular such corresponding to brain activity) the ongoing calculation of early warning indicators for seizures from measured data, and secondly in case of critical indicator values, the calculation of seizure-preventing interventions (based on a seizure model) and the ongoing implementation of these interventions by extracranial generation of suitable magnetic fields.

### RELATED APPLICATIONS

This application is a Continuation of PCT application Ser. No. PCT/EP03/03543 filed on Apr. 4, 2003 (which was published in German under PCT Article 21(2) as International Publication No. WO 03/084605 A1), which claims priority to German Application No. DE 102 15 115.6, filed Apr. 5, 2002, both applications being incorporated herein by reference in their entirety.

This application is related to U.S. application No.: (Attorney Docket No. 0001.0014US1 (US-5443)) filed on even date herewith by Oliver Holzner, entitled “Method and Apparatus for Electromagnetic Modification of Brain Activity,” which is also incorporated herein by reference in its entirety.

### BACKGROUND OF THE INVENTION

Known techniques for the prevention of epileptic seizures in vivo comprise the following approaches:

1) Research has been conducted with respect to the way, in which epileptic seizures evolve, primarily focused on brain slices (“in-vitro”) [8], [9].

2) Seizure-warning methods based on extra-cranial EEG-data have been described in principle, e.g. [1], [21].

3) Application of TMS (“transcranial magnetic stimulation”) for diagnostic purposes has been described in principle, also coupled with EEG, e.g. [2].

4) Application of TMS for intervention in cases of epilepsy consists of finding an epileptic focus based on medical experience of a medical doctor, imaging techniques, and/or trial and error, with subsequent attempts (using one- or two-coil systems) to produce seizures, e.g. ([5], [6], [10]).

5) Seizure-models, which describe prerequisites and features of collective neural dynamics exist as examples of recent physical theories like synergetics (for an overview see [7]).

WO 98/18394 describes a method, with which a magnetic stimulation of a test person will be conducted whilst his or her brain activity is measured using EEG. This known method is used for diagnostic purposes.

WO 01/21067 describes a method for early recognition of a forthcoming epileptic seizure. It is claimed that, using this method, a forthcoming epileptic seizure can be predicted hours or days in advance. In this method the brain activity of a patient is measured in different locations during and after an epileptic seizure. Using different nonlinear techniques, specific pairs of sensors will be singled out for a particular patient, with which seizures of this patient have been predicted particularly well (during a training period including several epileptic seizures). The necessity of ongoing re-adaptation of signal pairs emanating from sensor pairs requires further seizures of this patient. Essential elements of the method are training and adaption, which prevent successful seizure prevention, because the former require ongoing updating of data based on further seizures of the patient.

### SUMMARY OF THE INVENTION

The invention relates to a method and an apparatus for automatic non-invasive controlled or regulated, respectively, electromagnetic prevention of epileptic seizures in vivo.

The object of the present invention is to create a method and an apparatus for prevention of epileptic seizures.

Using a seizure model results in a reliable prevention of epileptic seizures. The present invention is based on the knowledge, that, using these models, processes leading to seizures can be quantified, and suitable control parameters can be specified, which makes a reliable seizure-prevention feasible.

In general according to one aspect, the invention features a method for non-invasive controlled or regulated, respectively, electromagnetic prevention of epileptic seizures in vivo. The method comprises automatic extracranial electromagnetic measurement of brain activity; automatic calculation of an early warning indicator for epileptic seizures; automatic calculation of an intervention instruction for preventing an impending seizure, triggered by a seizure warning by the early warning indicator, by means of a seizure model and the brain activity measured; and automatic intervention according to the intervention instruction, using controlled or regulated, respectively, extracranial generation of magnetic fields.

The above and other features of the invention including various novel details of construction and combinations of parts, and other advantages, will now be more particularly described with reference to the accompanying drawings and pointed out in claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings re not necessarily to scale; emphasis has instead been placed upon illustrating the principles of the invention. Of the drawings:

In the following the invention will be described by way of examples, referring to the appended drawings:

FIG. 1 shows a transmitter from the side.

FIG. 2 shows a transmitter from below.

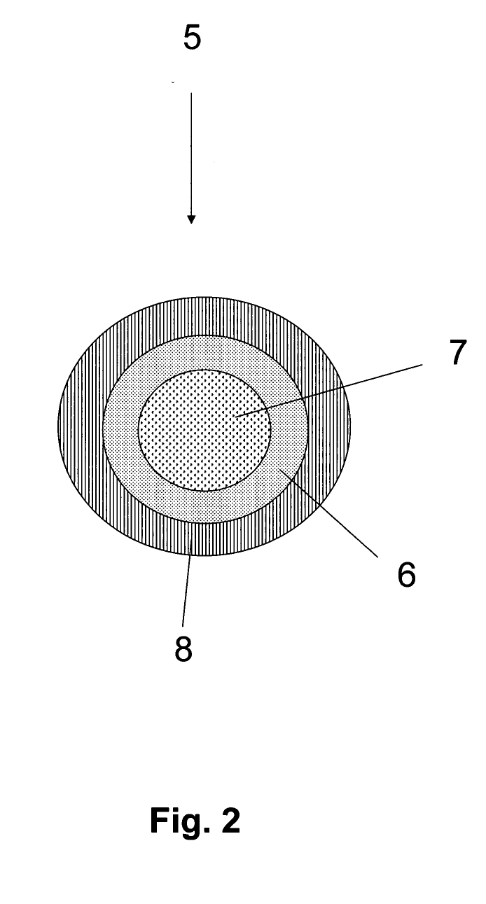

FIG. 3 shows a planar projection of drill-holes for sensors and transmitters displaying their locations within a helmet.

FIG. 4 shows a helmet with overhead suspension and chin-rest.

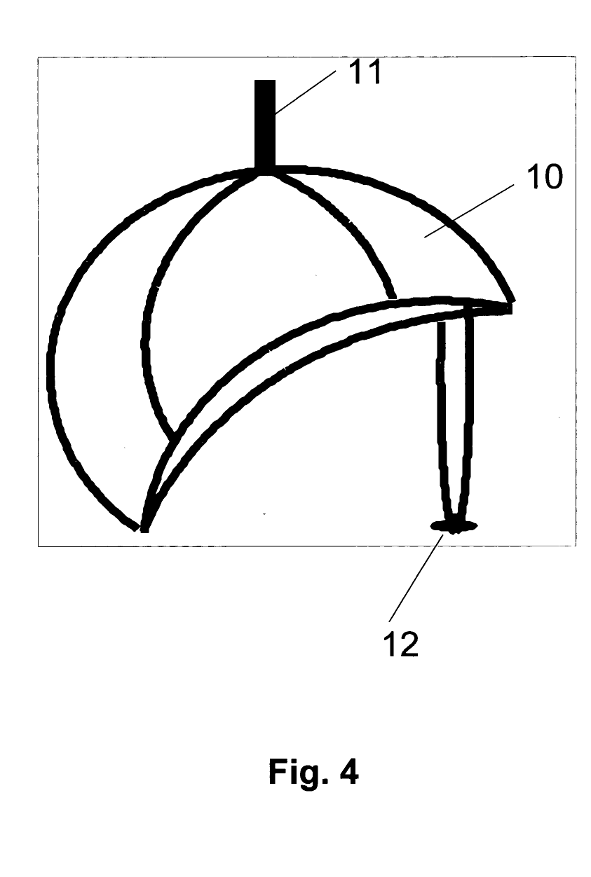

FIG. 5 shows another planar projection of drill-holes for sensors and transmitters displaying their locations within a helmet.

FIG. 6 shows an example for a time-series of measured EEG values from one sensor.

FIG. 7 shows the phase-space representation of parts of this time-series.

FIG. 8 shows a typical SNR-diagram (“signal-to-noise-ratio”).

### DETAILED DESCRIPTION OF THE PREFERRED EMBODIMENTS

Apparatus:

In accordance with the principles of the invention, an apparatus comprises, on the input side, a measurement system with devices for electromagnetic measurement, data processing, and data transfer, in a preferred embodiment of the invention e.g. including and EEG-cap with sensors, connections to amplifer, amplifier, connections to A/D-convertor, A/D-convertor, connections to the computer, power sources, and connections.

The apparatus comprises, on the output side, a control system with devices for the extracranial generation of magnetic fields, to be called “transmitters”,an apparatus for the transfer of control signals to transmitter signals, in a preferred embodiment of the invention e.g. including conducting coils, power sources, connections, D/A-converter.

Furthermore the apparatus includes a computer (PC or workstation) between input- and outside side, with software for implementation of the method described below in more detail.

Suitable sensors are EEG- or MEG-sensors. The MEG sensors comprise, e.g. a SQUID sensor element with suitable evaluation device for detecting a magnetic field, plus cooling device. EEG-sensors include two electrodes for measuring a difference in electric potential.

A sensor may contain electric and/or magnetic shielding with respect to its environment, which does not hamper its functionally (e.g. not shielded in the direction of the cranium of the patient, but shielded in the direction of the other transmitters, and/or sensors, and/or connecting cables).

Parts of the input side near the head may contain a plurality of sensors, distributed over those parts of the surface of the head, which are close to the brain. The plurality is called sensor grid.

The sensor grid includes fittings such that, after taking the sensor grid on and off several times, the sensors will resume their previous position with respect to the cranium of the respective patient. This is achieved, e.g., by fitting the sensor grid to the inside of a helmet, the inside of which is fitted to the cranial shape of the patient. The fitting of the sensor grid with respect to the cranium of the patient can also be supported by cameras, where the spatial position of the patient's cranium is calculated in real-time by using position data from several cameras.

A preferred embodiment of the invention of the input side is a partially ambulatory mode of operation, wherein data acquisition is performed via a portable sensor grid, which is connected with preprocessing units (to be carried in a rucksack, or as part of the patient's garments), and from where in a preferred embodiment a wirless connection to the computer unit is established.

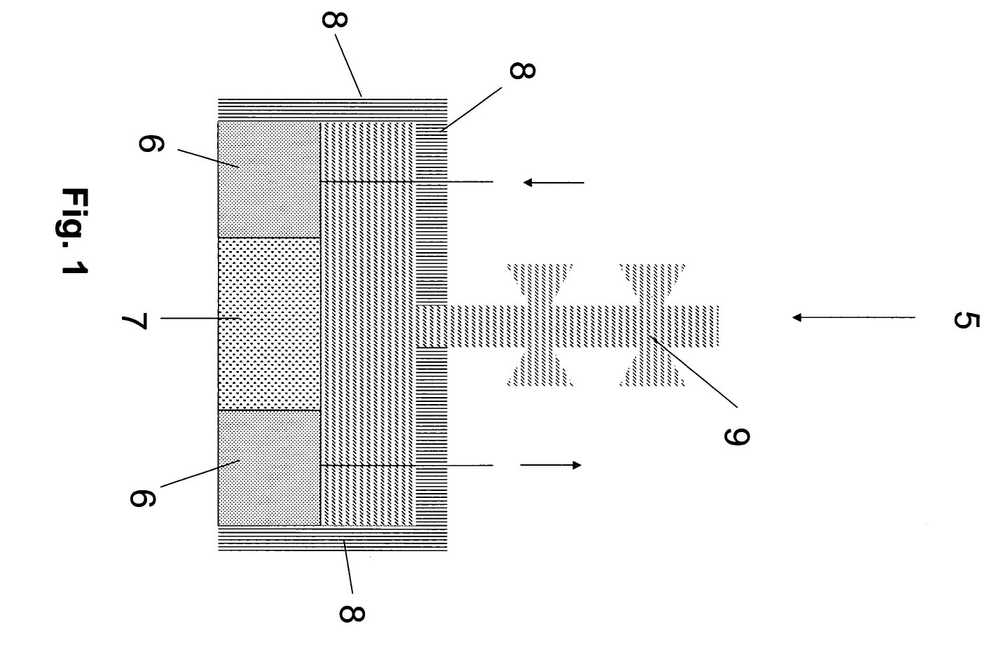

A transmitter 5 contains an induction coil 6 with para-, dia-, or ferromagnetic core 7, as shown from the side in FIG. 1, whereby arrows symbolize the direction of the induction current. The transmitter 5 has essentially a cylindrical shape, with sides and top being shielded (shield 8). At the unshielded bottom of the transmitter, pointing towards the cranium, adjacent coil 6 and core 7 are shown, which enable transmitter 5 to generate exogenous magnetic fields directed towards the cranium. On the back transmitter 5 a fitting element 9 is located, with which the transmitter 5 can be fitted into a helmet.

The extracranial transmitter 5 may be protected against deformation, e.g. by embedding the conducting parts into a suitable resin, or into insulating material.

Transmitter 5 may be provided with a cooling device.

In another embodiment of the invention, implanted electrodes, by which EEG can be measured, and by which currents can be transported into the brain, are used as sensors and/or transmitters. Connections with these electrodes and/or their interfaces with the computer and/or further measurement devices and/or the computer unit and/or power sources for the electrodes and/or the computer are also implantable, such that an ambulatory mode of operation becomes possible.

A preferred embodiment of the invention relating to parts of the control system near the head includes a plurality of transmitters, distributed intra- and/or extracranial. The plurality is called transmitter grid.

A preferred embodiment of the invention relating to the extracranial transmitter grid includes fittings with respect to the cranium of the patient, such that, taking the transmitter grid on and off several times, the transmitters will retake their previous relative position. This is achieved, e.g., by fitting the transmitter grid to the inside of a helmet, which is fitted to the cranial shape of the user.

Another preferred embodiment of the invention relating to the transmitter grid includes implanted electrodes.

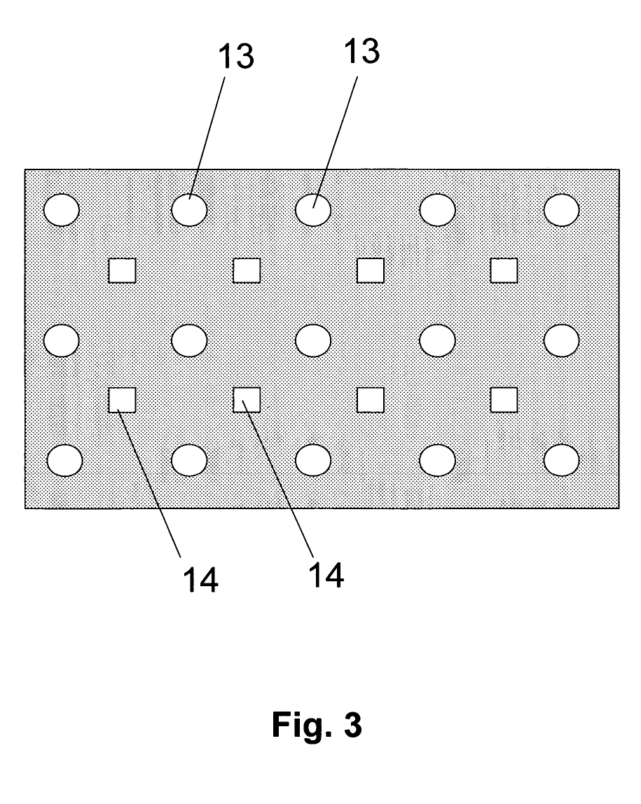

A preferred embodiment of the invention relating to the parts near the head of an extracranial measurement- and control-system comprises of a helmet 10, which fits the cranial shape of the respective patient, together with a cylindrical overhead suspension 11 with connection cables inside of it, and a chin rest 12. Sensor- and transmitter-grid on the inside of the helmet are fitted in such a way, that both grids are superposed, i.e. in the vicinity of each sensor there are sufficiently many transmitters, and vice versa. A planar projection of the superposition of transmitter and sensor grid is shown in FIG. 3 (drill-holes 13 for sensors are shown as circles, drill-holes 14 for transmitters 5 as squares). In the embodiment of the invention described the patient sits on an armchair with a neck-rest below helmet 10.

In an alternative embodiment of the invention, the sensor grid is intracranial, and the helmet contains the extracranial sensor grid.

In still another embodiment of the invention, both sensor-and transmitter-grid are intracranial.

In a preferred embodiment of the invention sensor-density, as well as sensor-configuration of an extracranial sensor-grid are adjustable. In another preferred embodiment of the invention the adjustment is performed automatically, controlled or regulated, respectively, by the intermediate unit.

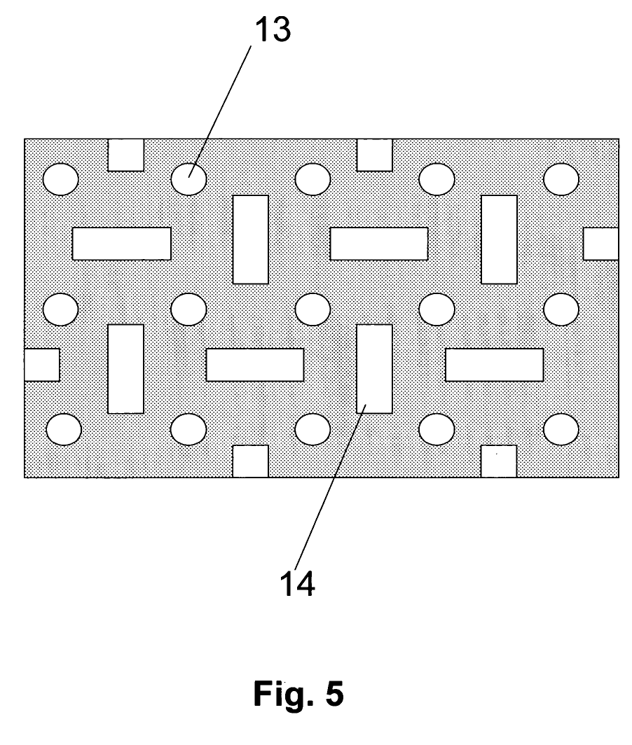

In a preferred embodiment of the invention transmitter-density, as well as transmitter-configuration of an extracranial transmitter-grid and/or the orientation of transmitters with respect to the cranium of the patient are adjustable. A planar projection of a mechanical fitting of this embodiment of the invention is shown in FIG. 5. Here drill-holes 13 for sensors are shown as circles, drill-holes 14 for transmitters as squares. It is possible, to fit transmitters 5 into the fittings of drill-holes 14, and/or tilt transmitters 5 with respect to the fittings. Amongst other coil configurations, all conventional coil configurations with their configurations and orientation of coils and electromagnetic fields can be emulated in this embodiment of the invention.

In a preferred embodiment of the invention, the apparatus contains conventional protection against power failure and/or voltage fluctuations.

In a preferred embodiment of the invention, on the computer unit the following methods are performed in real-time and automatically:
i) Ongoing calculation of the early-warning-indicator for seizures from input data
ii) In case the early warning indicator passes a threshold, calculation of an intervention instruction to prevent the seizure, as well as carrying out the intervention by means of generating magnetic fields using the transmitter(s).
iii) In case of return of the indicator into its normal range, and/or in case of exceeding a time limit: reduction of the intervention to zero.
iv) Conventional algorithms for removal of artifacts produced by exogenous magnetic fields (see, e.g. [2]), as well as removal of other artifacts (generated e.g. by muscle activity).

In a preferred embodiment of the invention, in addition to i)-iv), real-time and automatic procedures for optimization of sensor- and transmitter-density and for sensor- and transmitter-positioning are implemented on the computer.

Method:

1. EEG-measurement, preprocessing of measured data, and transfer digitized measured data including possible artifact removal are carried out on an ongoing basis using conventional methods. From measured data, the value of the empirically validated early warning indicator, which is used, is calculated automatically.

2. In case of an early seizure warning, an intervention instruction for the purpose of seizure prevention will be calculated (compatible with the seizure model used), which is carried out on an on going basis by means of generation of magnetic fields (B-fields) using transmitters. The details of the generation of magnetic fields (e.g. location, strength, orientation, frequency pattern, and/or others) are specified in the intervention instruction. Changes of the B-field generate intracranial induction potentials. Digital control of magnetic field generation according to the instruction is carried out using conventional methods. Applicable health recommendations for electromagnetic fields generated extracranial are known. They are followed automatically.

3. In case of the early warning indicator returning to its normal range and/or exceeding a time limit for the intervention, the intervention will be reduced automatically.

An early warning indicator is an index, calculated from electromagnetic brain activity data, which changes significantly before an epileptic seizure. For the purpose of the present invention, early warning indicators will be preferred, the change of which precedes the seizure by at least several minutes.

A suitable early warning indicator is the correlation of similarity indices of a predefined percentage of sensors, in case of diminishing similarity indices. The similarity index is known from [1] and a multitude of previous publications, e.g. [21]. The average early warning period claimed there is 325 seconds.

Another preferred embodiment of the invention relating to the method uses mutual information of similarity indices of a pre-defined percentage of sensors, in case of diminishing similarity indices. “Mutual information” is known as binary logarithm of the “probability of mutual occurrence of two random variables divided by the product of their individual occurrence probabilities”.

Another preferred embodiment of the invention relating to the method uses mutual information of similarity indices of a pre-defined percentage sensors, in case of diminishing similarity, combined with activation indicators (e.g. changes of body temperature characteristic for waking up, muscle movements, characteristic EEG patterns, and/or others). Thereby the possibility of blind arms caused by simultaneous multi-sensor changes caused by changes in alertness of the patient is reduced. Depending on the additional indicator used, there are additional device requirements (e.g. ongoing EMG measurements).

The examples for the calculation of early warning indicators given above do not require any training periods including epileptic seizures. The calculation of these early warning indicators uses a phase space representation of the normal state of the patient.

The early warning indicators listed above are robust with respect to noise and artifacts. For other, non-robust early warning indicators filtering and artifact removal methods need to be integrated into the procedure.

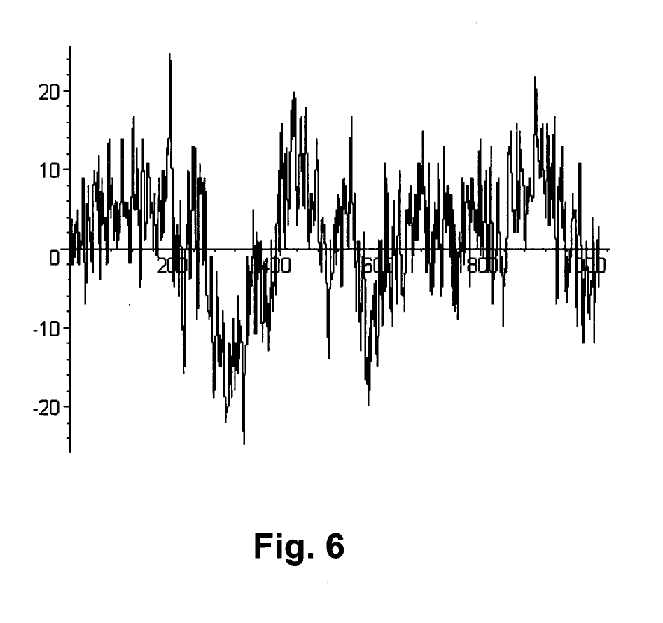

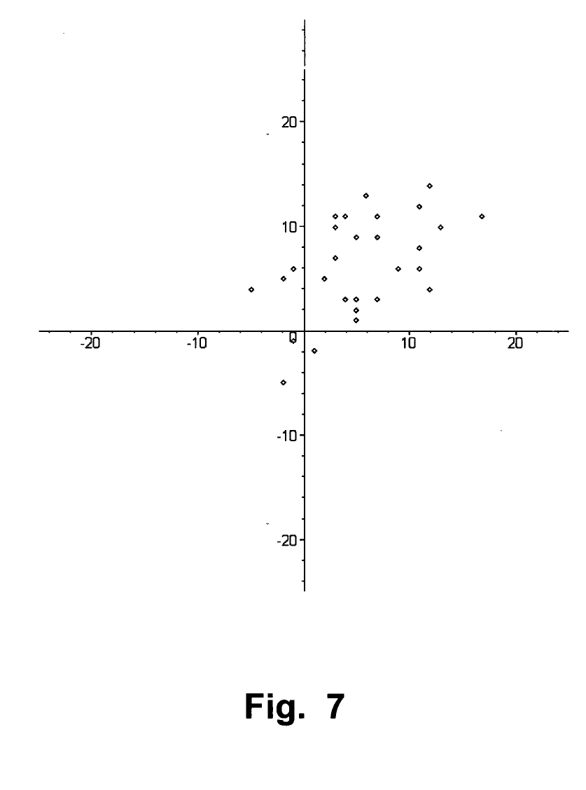

An example for phase space representation is given in FIGS. 6 and 7, whereby FIG. 6 shows 8 seconds of one channel of an EEG, at a sampling rate of 128 data points per second (x-axis represents time, y-axis voltage between electrode and reference electrode, in arbitrary units). FIG 7 shows a window of 32 data points from the time series of FIG. 6, starting with data point number 128, in phase space representation (x-axis potential difference of a data point at time t, y-axis value of a data point at time t−20). The method of how to embed a time series into a phase space is described comprehensively, e.g. in [3]. Underlying assumption is, that the one-dimensional signal (like in FIG. 6) is a projection of a higher-dimensional signal, which shall be restored. This higher-dimensional signal is shown in FIG. 7 in a two-dimensional representation.

A preferred embodiment of the invention relating to an early warning system for epileptic seizures can be a detection module with
1) a means for calculating, from each measurement channel, the similarity of the time series obtained in this channel with the time-series representing the normal state of each individual patient. Before using the detection module, the normal state of each patient needs to be sampled.
2) a means for giving a local warning signal for each measurement channel, in case said similarity should decrease below a threshold value.
3) a means for giving a global warning signal, if within a short period of time, several local warning signals for different channels are given.

To make the intervention reliable, seizure models are used. As seizure models the following models can be used: oscillator seizure model, chaos seizure model, synergetics seizure model, stochastic oscillator seizure model, stochastic chaos seizure model,stochastic synergetics seizure model.

These seizure models describe indexes relevant for epileptic seizures, which are calculated from the electromagnetic activities of neurons and/on neural populations. These indexes are e.g. chaoticity, calculated from the time series of potential differences between an EEG electrode and a reference via maximal Lyapunov exponents [12]. Other typical indexes are critical slowing down, critical fluctuations, similarity with a normal state in (meta-) phase space, etc. These indexes are expressed as actual numbers. E.g. chaoticity can, instead of by means of Lyapunov exponents, alternatively be expressed via embedding dimension [13], correlation dimension, Kullback-Leibler-entropy,etc. An oscillator seizure model is based on [3]. Here the neural populations described are limit-cycle-oscillators, which denotes that they may, depending on parameters, either oscillate or not oscillate. The interaction of neural oscillators is described in an interaction equation. Interaction is necessary condition for the emergence of a seizure. Prevention of seizures is based on decoupling of neural oscillators.

In the context of the present invention “neural oscillator” will be used as an equivalent to “limit-cycle-oscillator”. A specific case thereof are phase oscillators (see e.g. [22], where amplitude and phase are decoupled, and only the phase of an oscillator is considered further. In phase space, a limit cycle can be represented by an arbitary closed curve, a phase oscillator as a circle. A respective seizure model supposes an increase in 1-clusters compared to other clusters. This special case and related intervention methods (resetting plus entrainment, see e.g. [22]) cn not be successfully applied to limit-cycle-oscillators, where not even an often repeated hard reset with high amplitude results in a seizure prevention (in any case problematic, considering health limits for rTMS). Contary to this, interventions valid for limit-cycle-oscillators also work for phase oscillators.

A suitable interaction for the oscillator seizure model is weak coupling of neural oscillators. Seizures are accompanied by increase in the number of oscillating neural oscillators including an increase in mutual information with respect to the oscillation frequencies of these weakly coupled neural oscillators. A neural oscillator is localized ensemble of neurons, which is capable of oscillating and non-oscillating behavior. The dynamics of neural oscillator under interaction with other oscillators is given by

A suitable interaction for the oscillator seizure model is weak coupling of neural oscillators. Seizures are accompanied by an increase in the number of oscillating neural oscillators including an increase in mutual information with respect to the oscillation frequencies of these weakly coupled neural oscillators. A neural oscillator is a localized ensemble of neurons, which is capable of oscillating and non-oscillating behavior. The dynamics of a neural oscillator under interaction with other oscillators is given by

$${\dot{z}}_{i} = {g}_{i} ( {z}_{i} ) + ɛ {\sum }_{j = 1}^{n} {h}_{ij} \cdot {z}_{j}$$

ε<<1.

For every I between 1 and n, zᵢ is a neural oscillator. gᵢ is given by the well-known Wilson-Cowan equations ([3]). For the i-th neural oscillator, hᵢⱼ is the connection strength from zⱼ to Zᵢ. The coupling strength □ is empirically known to lie between 0.04 und 0.08. If changes in coupling strength and connection strengths are assumed to be slow compared to the time scale of a seizure, an intervention can be based on either a strong, as global as possible exogenous perturbation, with a possible transition from oscillation to non-oscillation, or an intervention via the function gᵢ. It is known from the theory of neural oscillators, that these interact only when they oscillate, and even then only in case of commensurate oscillation frequencies.

A preferred embodiment of the present invention relating to an intervention instruction compatible with the “seizure model with specific weak coupling between neural oscillators” is:

Force adjacent neural oscillators (which oscillate, before the intervention, with the same and/or commensurate frequencies) to incommensurate frequencies, which are factors of the original frequency or close to it (example: of adjacent oscillators oscillating at frequencies 3 Hertz and 15 Hertz, the second one is forced to oscillate with 5 Hertz; another example: both oscillate at 8 Hertz, therefore force one of them to oscillate at 7 Hertz). The forcing is performed with high amplitude magnetic fields at these frequencies. As adjacent neural oscillating at the same and/or commensurate frequencies imply the possibility of physiological connections between them, the emergence of a seizure will be prevented by forced incommensurateness, i.e. changes of gi, which block the possible and, even more so, the factual interaction between the oscillators and minimize their mutual information, and thereby prevents the emergence of a seizure. Procedural complexity allows for real-time calculation of all indexes needed.

Whether the attempt to make adjacent oscillators incommensurate is successful depends on how different they are and how minimal their mutual coupling is. In the limit of adjacent almost identical strongly coupled oscillators within the range of influence of the same transmitter, it is not possible to force then to different incommensurate frequencies. In this case it is sufficient to change groups of oscillators within the ranges of influence of the different transmitters to incommensurate frequencies, in order to prevent the seizure. This also prevents the information of 1-clusters within the intersection of the ranges of influence of several transmitters, and prevent the emergence of traveling waves.

Another advantageous embodiment of the invention relating to an intervention instruction compatible with the “seizure model with specific weak coupling between neural oscillators” is: step 1: chaotize the neural oscillators [14](e.g. by time-delayed feedback with bias), subsequent step 2: stabilize the neural oscillators on orbits with incommensurate frequencies, using conventional methods. As known from [4] step 2 of this procedure has been demonstrated in vitro to block spatial spreading of seizures. However the algorithm used there (“OGY”) is not suitable for real-time applications due to its demands on storage capacity and processing speed.

In the stochastic oscillator seizure model, in addition to the abovementioned model certain parameters are assumed to be stochastic. The methods described above are also applicable (e.g. [15]).

The chaos seizure model is based on normal brain activity (as measured at every sensor) having a minimum of chaoticity. Seizures go along with a decline of chaoticity, which is simultaneous for all sensors. Seizure prevention is based on maintenance of a certain minimum of chaoticity ([4] and [16]).

In the stochastic chaos seizure model high-dimensional influences augment the low-dimensional deterministic change of electromagnetic indexes seizures prevention measures resemble those of the chaos seizure model.

The synergetics seizure model is based on the fact, that brain activity is governed by a small number of degrees of freedom, called order parameters [17], subject to circular causality: order parameters are caused and determined by cooperation of neurons, at the same time order parameters determine the macroscopic behavior of the system. An epileptic seizure corresponds to a phase transition, which goes along with critical slowing down and critical fluctuations. Seizure prevention is performed by prevention of this phase transition (e.g. by control of bifurcation points, see e.g. [18]).

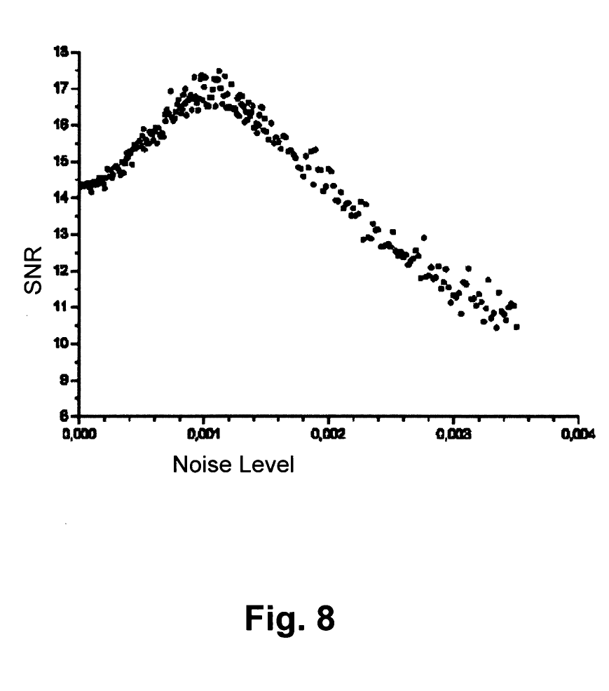

In the stochastic synergetics seizure model, the synergetics seizure model is augmented by phenomenological stochastic forces (“Langevin approach”). In addition to said methods of seizure prevention stochastic resonance [20] and its opposite (noise-drowning) are possible: it is known that in systems with stochastic components, depending on a noise amplitude (e.g. for Gaussian white noise), signals may be generated (“Coherence Resonance”), respectively the signal-to-noise-ratio (SNR) may be increased (“Stochastic Resonance”) or deceased (which shall here be noted as “noise drowning”). The typical shape of SNR is shown in FIG. 8 (x-axis: noise amplitude, y axis SNR).

By means of these models, an intervention instruction will be calculated, which describes the magnetic field to be generated. This description is e.g. given by location, strength, orientation, frequency pattern, and/or other parameters of magnetic field (B-field). With this magnetic field the electromagnetic activities of neurons and/or neural populations will be changed in a way suitable for preventing and impending epileptic seizure.

Applying one or several models results in a reliable prevention of epileptic seizures. The present invention is based on the knowledge, that with these models the processes leading to epileptic seizures are captured in a quantitative way, specifying suitable control parameters, such that a reliable prevention is feasible.

The preferred embodiment of the invention comprises an intervention module, which in a plurality of models is suitable for preventing the seizure. E.g. in case of high transmitter density transmitters can be classified into three classes:
class 1 for chaotizing,
class 2 for incommensurate stabilizing,
class 3 for noise drowning,

such that in the vicinity of each transmitter of one class transmitters of the other classes are located.

In addition to other effects class 1 fulfills requirements of chaos seizure models, class 2 of oscillator seizure models, class 3 of models with stochastic components. In this case requirements of synergetics seizure models are automatically fulfilled, by destruction of master modes by frequency shifts, and at the same time prevention of slave modes developing into master modes by noise drowning. Requirements of phase oscillator seizure models are also fulfilled automatically, as 1-cluster-states are prevented (incommensurateness prevents phase-locking, noise-drowning prevents higher modes). Requirements of chaos seizure models are also fulfilled automatically, due to class 1and class 3(noise=high dimensional chaos).

In said methods brain activity can be measured wither during or immediately after intervention, resulting in closed loop control, because from brain activity measured the early warning indicator and, if necessary, a further intervention instruction are calculated.

A preferred embodiment of the invention related to reduction of intervention is simultaneous reduction of all magnetic fields generated.

A preferred embodiment of the invention related to reduction of intervention is smooth reduction of the density of transmitters, which generate the magnetic fields (reduction as spatially homogenous reduction for all transmitters and/or by switching off a percentage of transmitters).

A preferred embodiment of the invention related to reduction of intervention is a spatially localized reduction or switching off of several transmitters, with gradual extension of the area, in which the reduction or switching off takes place.

It is not necessary to intervene with field strengths of 1-2 Telsa per coil, as conventionally used in TMS. “Ongoing” is defined as “continuous” or “repeated after suitable periods of time”. Monitoring electromagnetic health limits is automatically carried out on an ongoing basis.

The purpose of the invention is not to cure epilepsy, but to prevent epileptic seizures during the application period of the invention, without necessity of medical attention or usage of drugs. This minmizes adverse consequences of the disease, as well as side effects, at the same time clearly reducing costs. Furthermore an ambulatory application of the invention is possible, which results in further cost reduction cost reduction as well as strongly improved mobility of the patients.

With the method of the present invention,described above, epileptic seizures are prevented. Furthermore, with a similar, but more general proactive method (instead of the reactive prevention of epileptic seizures based on seizure models) other behavioral targets, preferably for healthy persons, can be reached using behavioral models in conjunction with general brain activity models. The general method includes the interactive determination of non-observables of the models, the calculation of a-priori unknown intervention instructions, as well as the selective implementation of the intervention instruction, accompanied by the simultaneous prevention of undesired spreading. It is the purpose of this variant of the invention to stabilize or modify the behavior of a person according to his/her wishes and/or stabilize the modification in a reliable way. This method can be carried out with an apparatus similar to the one described above.

References

[b 1] “Anticipation of epileptic seizures from standard EEG recordings”, Le Van Quyen M, Martinerie J, Navarro V, Boon P, D'Have M, Adam C, Renault B, Varela F, Baulac M, The Lancet 2001 Jan. 20; 357: 183-8
[2] WO 98/18384 A1
[3] “Excitatory and inhibitory interaction in localized populations of model neuron”, Wilson H R, Cowan J D, Biophysical Journal 1972; 12: 1-22
[4] “Controlling chaos in the brain”, Schiff S J, Jerger K, Duong D H, Chang T, Spano M L & Ditto W L, Nature 1994 Aug. 25; 370: 615-620
[5] “Transcranial magnetic stimulation in patients with epilepsy”, Dhuna A, Gates J, Pascual-Leone A, Neurology 1991 July; 41: 1067-1071
[6] “Epileptic seizures triggered directly by focal transcranial magnetic stimulation”, Classen J, Witte O W, Schlaug G, Seitz R J, Holthausen A, Benecke R, Electroencephalograph and Clinical Neurophysiology 1995; 94: 19-25
[7] “Epilepsy: multistability in a dynamic disease”, Milton J G, in “Self-organized biological dynamics and nonlinear control”, ed. Wlleczek J, Cambridge University Press 2000
[8] “Electric field suppression of epileptiform activity in hippocampal slices”, Gluckman B J, Neel E J, Netoff T I, Ditto W L, Spano M L, Schiff S J, Journal of Neurophysiology 1996 December; 76 (6):4202-4205
[9] “Coupled intra- and extracellular Ca2+ dynamics in recurrent seizure-like events”, Szilagy N, Kovacs J, European Journal of Neuroscience 2000; 12: 3893-3899
[10] “Deliberate Seizure Induction With Repetitive Transcranial Magnetic Stimulation in Nonhuman Primates”, Lisanby S H, Luber B, Sackeim H A, Finck A D, Schroeder C, Arch Gen Psychiatry 2001; 58: 199-200
[11] WO 01/21067
[12] “How to Extract Lyapunov Exponents from Short and Noisy Time Series”, Banbrook M, Ushaw G, McLaughlin S, IEEE Transactions on Signal Processing 1997; 45: 1378-1382
[13] “Determining embedding dimension for phase-space reconstruction using a geometrical construction”, Kennel M B & Brown R & Abarbanel H D I, Physical Review A 1992, 45 (6): 3403-3411
[14] “Chaotification via arbitrary small feedback controls: theory, method, and applications”, Wang X F, Chen G; International Journal of Bifurcation and Chaos 2000; 10 (3): 549-570
[15] “Controlling Nonchaotic Neuronal Noise Using Chaos Control Techniques”, Christini D J, Collins J J, Physical Review Letters 1995 Oct. 2; 75 (14): 2782-2785
[16] “Anticontrol of chaos in continuous-time systems via time-delay feedback”, Wang X F, Chen G, Yu X; Chaos 2000 December; 10 (4): 771-779
[17] “A derivation of macroscopic field theory of the brain from quasi-microscopic neural dynamics”, Jirsa V K, Haken H, Physica D 1999, 99: 503-526
[18] “Controlling Bifuration Dynamics via Chaotification ”, Wang S F, Chen G; proposed paper for CDC 2001
[19] “Impact of noise on a field theroretical model of the human brain”, Frank T D, Daffertshofer A, Beek P J & Haken H, Physica D 1999, 127: 233-249
[20] “Functional Stochastic Resonance in the Human Brain: Noise Induced Sensitization of the Human Baroflex System”, Hidaka I & Nozaki D & Yamamato Y, Physical Review Letters 2000 Oct. 23; 85 (17) 3740-3743
[21] “Anticipating epileptic seizures in real time by non-linear analysis of similarity between EEG recordings”, Le Van Quyen M, Martinerie J, Baulac M, Varela F, Neuroreport 10 (1999): 2149-2155
[22] “Effective desyncronization with a stimulation technique based on soft phase resetting”, Tass P, Europhysics Letter 57 (2), 2002: 164-170

While this invention has been particularly shown and described with references to preferred embodiments thereof, it will be understood by those skilled in the art that various changes in form and details may be made therein without departing from the scope of the invention encompassed by the appended claims.

1. Method for non-invasive controlled or regulated electromagnetic prevention of epileptic seizures in vivo, comprising the following steps:
automatic extracranial electromagnetic measurement of brain activity;
automatic calculation of an early warning indicator for epileptic seizures;
automatic calculation of an intervention instruction for preventing an impending seizure, triggured by a seizure warning by the early warning indicator, by means of a seizure model and the brain activity measured; and
automatic intervention according to the intervention instruction, using controlled or regulated extracranial generation of magnetic fields.

2. Method according to claim 1, wherein a seizure model is used, which is based on indicators calculated from the electromagnetic activities of neurons and/or neural populations, and which are relevant for epileptic seizures.

3. Method according to claim 2, wherein a seizure model of the following group is used, comprising an oscillator seizure model, a stochastic oscillator seizure model, a chaos seizure model, a stochastic chaos seizure model, a synergetics seizure model, a stochastic synergetics seizure model.

4. Method according to claim 1, wherein that measurement of brain activity and calculation of early warning indicators is carried out on an ongoing basis.

5. Method according to claim 1, wherein an intervention instruction is carried out by generating extracranial magnetic fields.

6. Method according according to claim 1, wherein brain activity is measured during or immediately after an intervention.

7. Method according to claim 1, wherein by controlled or regulated reduction of the intervention in case of the early warning indicator returning to a normal range and/or in case of exceeding a time limit.

8. Apparatus for automatic non-invasive controlled or regulated, respectively, electromagnetic prevention of epileptic seizures in vivo, the apparatus comprising:
a measurement device with at least one sensor to measure electro-magnetic brain activity,
means for determining an early warning indicator for detecting impending epileptic seizures in advance,
means for calculating an intervention instruction based on a seizure model and brain activity measured, and
means for implementing the intervention instruction, using at least one transmitter for generating a magnetic field.

9. Apparatus according to claim 8, wherein the measurement device comprises several sensors, which constitute a sensor grid.

10. Apparatus according to claim 8, wherein the sensors are located on an EEG-cap.

11. Apparatus according to claim 8, wherein the mechanism for the implementation of the intervention device comprises several transmitters, which constitute a transmitter grid.

12. Apparatus according to claim 8, wherein a computer, is used, on which a software module for the implementation of the method according to one of the claims 1 to 7 is stored.

13. Apparatus according to claim 8, wherein electric and/or magnetic shielding for every sensor and every transmitter is used.

14. Apparatus according to claim 8, wherein the position of the measurement device with respect to the cranium of the patient is fixed, such that, after taking the sensor grid on and off several times, the sensors will resume their previous relative positions.

15. Apparatus according to claim 8, wherein the measurement device is mechanically decoupled from the other parts of the apparatus, such that the patient may carry the measurement device around.

16. Apparatus according to claim 8, wherein the means for carrying out the intervention instruction are planned to be locked to a fixed position with respect to the cranium of the patient.

17. Apparatus according to claim 8, wherein sensors and transmitters are localized on the inside of a helmet which fits the shape of the cranium of respective patient.

18. Apparatus according to claim 9, wherein the sensor grid and transmitter grid are superposed, such that there are transmitters in the vicinity of each sensor, and sensors in the vicinity of each transmitter.

19. Apparatus according to claims 11, wherein fittings in the transmitter grid are provided, such that additional transmitters might be fitted thereto, such that the transmitter density of a transmitter grid may be changed locally and/or the angle of the individual transmitters with respect to the cranium of the patient may be modified.
